(19) United States
(12) Patent Application Publication
(10) Pub. No.: US 2022/0132941 A1
(43) Pub. Date: May 5, 2022

(54) ARTICLE OF CLOTHING AND METHOD FOR CONTROLLING CELLS USING SUCH AN ARTICLE OF CLOTHING

(71) Applicant: Max BOYSSET, Bonvillards (CH)

(72) Inventor: Max BOYSSET, Bonvillards (CH)

(21) Appl. No.: 17/431,054

(22) PCT Filed: Feb. 14, 2020
(86) PCT No.: PCT/EP2020/053831

§ 371 Date: Aug. 13, 2021

(30) Foreign Application Priority Data
Feb. 15, 2019 (EP) 19157593.5

(51) Int. Cl. A41C 3/00; A41D 1/00; A61B 8/08; A61B 8/00

(52) U.S. Cl. CPC A41C 3/0064; A61B 8/4494; A61B 8/0825; A41D 1/002

(57) ABSTRACT

The present invention relates to a piece of clothing (10) intended to be worn by a user so as to cover a part of the user's body. This piece of clothing (10) comprises a flexible clothing element (11) covering said part of the user's body as well as a generator (14) of electrical signals and at least one transducer (12) integral with said flexible clothing element (11). Said at least one transducer is arranged to convert said electrical signals from the electrical signal generator (14) into ultrasounds and to transmit the ultrasounds generated by this transducer (12) to said part of the user's body covered by this flexible clothing element. The piece of clothing is characterised in that said at least one transducer (12) is arranged to emit ultrasounds having at least two different acoustic intensities comprised in at least two distinct intensity ranges.

The present invention further relates to a method of monitoring biological cells of a user, which method uses the piece of clothing as described above. The method comprises the following steps:
selecting an acoustic intensity from at least two distinct acoustic intensity ranges;
energising the transducer (12) so as to emit ultrasonic waves according to the selected acoustic intensity;
emitting ultrasonic waves from the transducer (12), said ultrasonic waves having an acoustic intensity within said selected intensity range.

### TECHNICAL FIELD

The present invention relates to an item of clothing intended to be worn by a user so as to cover a part of the body of the user.

Such an item of clothing may be used in the context of prevention, detection and/or treatment of certain pathologies such as cancers. The item of clothing may be an undergarment, for example.

The present invention also relates to a method for monitoring biological cells of a user.

### PRIOR ART

Currently, there are few effective and proven means for the prevention of certain pathologies such as cancers and in particular breast cancer, testicular cancer or colorectal cancer. Generally, prevention is limited to minimizing risk factors such as smoking, alcohol, UV radiation or unhealthy food. The disease is managed at the time it is declared and detected, which often requires drastic management techniques, which can be painful and particularly distressing for the patient.

It would be desirable to have a solution for cancer prevention that is unobtrusive, easy to implement and present as little disturbance as possible to the user.

For the detection of cancer, especially breast cancer, mammography is usually recommended at regular intervals, for example every year or every two years, for women of a certain age. Because mammograms are painful, inconvenient and unpleasant, not all women are willing to have them done regularly. In addition, mammograms require an appointment and travel to the site of the checkup, which is a burden that does not encourage all women to have regular checkups.

If cancer develops shortly after an examination, it may take a long time for a new examination to be performed and for the cancer to be detected. It is well known that the earlier a cancer is detected and treated, the greater the chances of recovery.

There is therefore a need for a simple, painless and regularly usable means for detecting breast cancer in particular, but also for other cancers.

Regarding the treatment of cancers, in particular breast cancers, the means of treatment are generally heavy and very invasive. These treatments include radiotherapy, chemotherapy and/or removal of the breast. There are also targeted therapies, but these are only possible for certain specific types of cancer.

Because these therapies are performed in a hospital setting, patients must travel and are treated for a relatively short period of time with relatively high doses of drugs. These high doses of drugs result in side effects that can be significant and particularly unpleasant.

Thus, there is a need for therapy that is as minimally invasive as possible, using smaller doses of therapeutics, but spread over a longer period of time so as to minimize side effects.

Publications US 2013/317364 and US 2013/317364 both describe, an item of clothing made in the form of a bra and intended for the early detection of a breast tumor.

For this purpose, the bra comprises a signal generator, for example for ultrasounds, and signal receivers. It also includes a device for processing the signals received by the receivers.

When the bra is worn by a user, the signal generator emits signals towards the user's breasts. These signals are scattered and reflected by the biological tissues of the breasts, and some of them are picked up by the receivers of the bra. The thus captured signals are processed by the processing device to determine whether the user's breasts contain abnormal biological tissue such as tumors or cysts.

If such abnormal biological tissue is detected, an alarm is triggered to inform the user of the presence of such an abnormal element and to indicate that medical consultation is recommended.

It is clear that the bras described in these documents are only useful and usable when abnormal tissue such as a tumor or cyst has formed. Such a bra is useless in the context of cancer prevention since, in the absence of abnormal tissue, it provides no information.

Similarly, such a bra is also useless for cancer treatment. Indeed, with the function of the bra being only to inform in case of detection of abnormal tissue, it would have no use if the presence of such abnormal tissue is already known.

There is therefore a need for an item of clothing that can be used not only to detect the existence of unhealthy cells, but also to prevent the appearance of such unhealthy cells and/or to treat such unhealthy cells after their appearance.

### DESCRIPTION OF THE INVENTION

Such an item of clothing is described in the present invention.

The purpose of the invention is achieved by an item of clothing intended to be worn by a user so as to cover a part of the body of the user, the item of clothing comprising a flexible clothing element covering said part of the body of the user, said item of clothing further comprising an electrical signal generator and at least one transducer integral with said flexible clothing element, said at least one transducer being arranged to convert said electrical signals from the electrical signal generator into ultrasounds and to transmit the ultrasounds generated by the transducer to said part of the user's body covered by the flexible clothing element, the item of clothing being characterized in that said at least one transducer is arranged to emit ultrasounds having at least two different acoustic intensities comprised in at least two distinct intensity ranges.

The purpose of the invention is also achieved by a method of monitoring biological cells of a user by means of an item of clothing intended to be worn by said user so as to cover a part of the body of the user, the item of clothing comprising a flexible clothing element covering said part of the body of the user, said item of clothing further comprising an electrical signal generator and at least one transducer integral with said flexible clothing element, said at least one transducer being arranged to convert said electrical signals from the electrical signal generator into ultrasounds and to transmit the ultrasounds generated by the transducer to said part of the user's body covered by the flexible clothing element, the method being characterized in that it comprises the following steps:
selecting an acoustic intensity from at least two distinct acoustic intensity ranges;
providing power to said at least one transducer so as to emit ultrasonic waves according to the selected acoustic intensity;
emitting ultrasonic waves from said at least one transducer, said ultrasonic waves having an acoustic intensity within said selected intensity range.

The item of clothing of the invention makes it possible to implement a mode of prevention of diseases, and in particular of cancers, without the user being disturbed or bothered. This prevention can be carried out without any discomfort or constraint for the user, in particular at home or at any moment of his or her daily life. This makes it possible to carry out prevention over long periods of time, unlike what would be possible, for example, if prevention had to be carried out in a specialized environment such as a hospital. Because of this long duration, prevention can be all the more effective.

The item of clothing can be adapted to the type of prevention desired and to the part of the user's body that the person concerned wishes to take into account. The item of clothing may in particular be a bra, underpants, tights, an undershirt, a tank top, a hood or cap, for example.

The item of clothing of the invention can also be used for the detection of abnormal cells. This detection can be done on a very regular basis, for example once a day or once a week, without the wearer of the item of clothing even noticing, i.e. without any discomfort or inconvenience to the user.

This daily or weekly detection, or any other chosen frequency, makes it possible to compare the results obtained on a certain day with the results obtained during previous controls and thus makes it possible to follow the evolution of the user's body part. It is thus possible to very quickly detect an abnormal evolution or modification and to act accordingly.

The item of clothing, or garment, can also be used for curative purposes. In this case, it can be specially adapted to the user, to his or her pathology and to the areas that the user wants to treat.

Since the item of clothing can be worn by the user in the same way as a conventional garment and does not present any discomfort, it is possible to wear the garment for a long period of time during the day, typically several hours a day, every day. This allows for a much lower dosage of treatment than when the treatment is applied for a very short period of time, such as one hour per week, which would typically be the case in a hospital setting.

The side effects that can be caused by high doses of treatment can be reduced or eliminated by applying lower doses for a longer period of time. The fact that the device of the invention can be used for very long periods of time compared to conventional treatments opens the door to different treatments and in particular to treatments that would have no effect or a very weak effect if they were applied for a short period of time. These treatments or therapies can potentially have no or lesser side effects than conventional therapies.

### BRIEF DESCRIPTION OF THE DRAWINGS

The present invention and its advantages will be better understood with reference to the appended figures and the detailed description of a particular embodiment, wherein:

FIG. 1 schematically illustrates a particular embodiment of an item of clothing according to the invention;

FIG. 2 is a schematic cross-sectional view of a portion of the garment of FIG. 1;

FIG. 3 represents a first mode of use of the item of clothing of the invention;

FIG. 4 illustrates a second mode of use of the item of clothing of the invention;

FIG. 5 is a variant of the embodiment illustrated in FIG. 4; and

FIG. 6 represents a third mode of use of the item of clothing of the invention.

### EMBODIMENTS OF THE INVENTION

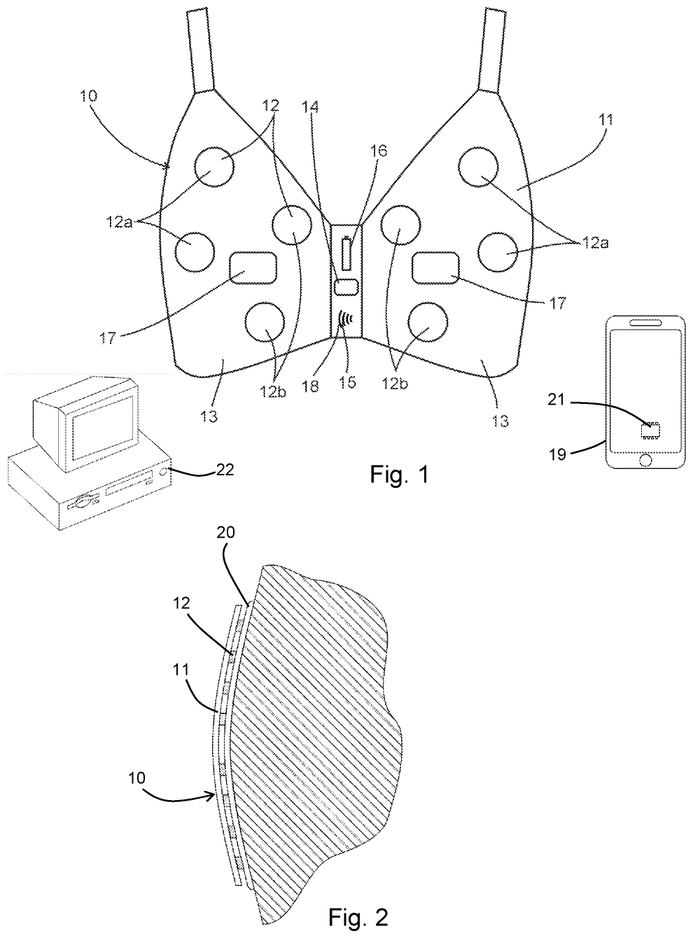

With reference to FIGS. 1 and 2, the item of clothing 10 according to the invention is shown in the form of a bra. However, it could have other shapes depending on the intended purpose of this item of clothing, or garment.

The item of clothing of the invention comprises a flexible clothing element 11 made generally of a fabric and intended to conform to the shape of the user's skin, to be in proximity to the user's skin and to cover a part of the user's body.

The item of clothing 10 comprises at least one ultrasound transmitter 12a, attached to the flexible clothing element 11. The item of clothing also comprises at least one receiver 12b of waves reflected by biological tissues of the wearer of the item of clothing, these waves coming from the reflection of the ultrasonic waves emitted by said at least one transmitter 12a.

In an advantageous embodiment, each transmitter 12a is formed by a transducer 12 having the function of transforming electrical signals into ultrasonic waves. Each receiver 12b is also formed of a transducer 12 having the function of transforming waves reflected from the user's biological tissues into electrical signals. These transducers may be physically the same elements alternately acting as transmitters and receivers, or they may be of the same nature, with some of them being used as transmitters and others as receivers, or they may be of different nature, with transducers of one type being used as transmitters and transducers of another type being used as receivers.

These transducers 12 are preferably elements of relatively small size and are made to be as unobtrusive as possible to the user.

In the practical case where the item of clothing is a bra, the bra is formed by two cups 13 that each carry at least one transducer 12. In the example shown in FIG. 1, each cup carries four transducers 12 arranged in different locations on the cups. The transducers may be curved and/or flexible, so as to best fit the shape of the bra and not be disruptive to the wearer.

The item of clothing of the invention includes an electrical signal generator 14 connected to the transducers 12. This signal generator 14 may be held in place by the flexible clothing element 11 or, on the contrary, may be arranged next to the flexible element. In the illustrated embodiment, the electrical signal generator 14 is arranged on the flexible clothing element 11, between the two cups 13.

The signal generator 14 is in charge of activating the different transducers 12 or not activating them, according to operations described in more detail below.

The transducers 12 are for receiving electrical signals from the signal generator 14 and to transform these electrical signals into ultrasounds. In this embodiment, the transducers act as transmitters. The generated ultrasounds may have different frequencies, different amplitudes, different powers or different acoustic intensities among others. It is possible to arrange for all the transducers to emit ultrasounds with the same characteristics or on the contrary, to emit ultrasounds with different physical characteristics.

The electrical signal generator 14 may be arranged to be adjustable so as to modify at least some characteristics of the waves generated by the transducers, under the control of the generator.

According to an embodiment, the ultrasounds used may have a frequency of between 600 kHz and 7 MHz and an acoustic intensity varying from 0.01 to more than 10 W/cm² depending on the selected mode of use.

According to a concrete embodiment, the transducers 12 are made of piezoelectric elements having a resonance frequency of about 2 MHz. They are made of PZT (Lead Titano-Zirconate) and have a size of 5×5 mm and a thickness of 1 mm. Each cup has 512 piezoelectric transducers, which allows the detection of a cluster of cells smaller or close to 1 mm in diameter. Such a size corresponds to an early stage of the cancer cell cluster.

Once the ultrasounds are generated by the transducers 12 or piezoelectric elements, they must be transferred to the human body. The acoustic impedance of the human body and the transducers is 1.5 MRayl and 35 MRayl, respectively. This difference in impedance makes the transmission of ultrasound between the two media very inefficient. With typical values of the acoustic impedance of the two media, the transmission coefficient is less than 0.1%, which means that a large portion of the ultrasonic waves is lost at the interface. A matching layer between the transducers and the skin is advantageously used to match the impedance.

A matching layer with an impedance of about 10 MRayl is optimal in a three-layer system, namely the transducers, the impedance matching element and the user's skin.

As with conventional ultrasound examinations, it is possible to apply an impedance matching gel directly to the user's skin. However, this solution is not optimal in the case of an item of clothing because of the requirement for ease of use and to avoid having to clean up the gel after use. However, this solution can still be used in practice.

A preferred solution is the use of a solid, flexible impedance matching element 20 that can be placed in the item of clothing, as shown in FIG. 2.

In a specific embodiment for making an impedance matching element, two methods have been practiced. The first is to create a PEG (poly(ethylene glycol) diacrylate) based hydrogel with adjustable properties to achieve an impedance of about 10 MRayl. To obtain a flexible hydrogel with a diameter of about 20 cm and a thickness of 5 mm, the following concentrations were used: PEGDA700 (12.5 g. 700 g. mol⁻¹); H2O (37.5 g) and 500 μL of a photo-initiator (2-Hydroxy-2-methylproplophenone, 97%) and a curing time of 30 minutes under UV light after degassing with nitrogen.

The second method tested uses silicone. To obtain a solid gel, the silicone must be placed in a polymer network. For this purpose, a PDMS (Polydimethylsiloxane) gel was made with a silicone elastomer (35 g) and a silicone elastomer crosslinker (3.5 g), cured for 10 h at 70° C. after degassing under vacuum.

A support material is attached to the electrode opposite the PDMS gel to prevent excessive transducer vibration. The reduction of excessive vibration implies that the transducer generates ultrasound waves with a shorter pulse length, improving the axial resolution in the images. However, a trade-off between axial resolution and sensitivity, which is related to the output voltage, must be made. The axial resolution gives the spatial resolution and the sensitivity level gives the noise level in the final image.

The clothing part 10 of the invention may also include an instruction receiver 15, which may be integrated with the electrical signal generator 14 or may be separate from it. This instruction receiver 15 is arranged to receive instructions from, for example, an application managed by an external device 19, such as a smartphone hosting a dedicated application. When an instruction message is received by the instruction receiver 15, it is processed and then transmitted to the signal generator 14, which will in turn activate the transducer(s) 12 according to the content of the received message. Such a message may, for example, indicate which of the transducer(s) 12 are to be activated, among all the available transducers, what is the amplitude, intensity and/or power of the ultrasonic waves to be generated, possibly their frequency, as far as the frequency can be adjusted, the duration, cycle parameters, etc.

As mentioned, the instruction receiver 15 can receive instructions from the device 19 external to the garment. However, it can also receive information from the garment itself. This information may in particular relate to the transducers 12 and indicate which transducers are active, the amplitude of the generated ultrasonic waves, their frequency, their intensity, the exposure time, cycle parameters, etc.

The item of clothing 10 may also include a power supply 16, typically implemented as a battery that may also be placed on or adjacent to the item of clothing 10. According to a preferred embodiment, this battery can be charged by means of a USB type connection and have a battery life, if possible, of at least 24 hours.

The item of clothing of the invention may further comprise one or more measuring devices 17 intended to measure certain parameters related to the person wearing the item of clothing. As an example, the measuring device 17 could measure the temperature at certain locations of the user's body in contact with the item of clothing.

The information sent to the instruction receiver 15 may also come from the measuring device 17 and relate to the parameters measured by that measuring device.

The instruction receiver 15 can include a communication module 18 arranged to transmit the collected information to an external device such as a memory, a communication device or a communication network in particular.

Once emitted by the transducers acting as transmitters, the ultrasonic waves pass through the impedance matching element 20 and propagate in different biological tissues. The different densities of the different biological tissues correspond to different impedances. These impedance differences create an echo that is picked up by the transducers. In this context, the transducers 12 act as receivers 12b. It should be noted that the transducers used as transmitters 12a can also be those used as receivers. It is also possible to use different transducers as transmitters and receivers, these transducers being however realized in the same way. It is also possible to use different transducers as transmitters and receivers, these transducers being made in different ways depending on their role. It is also possible to use different transducers as transmitters, depending on the physical characteristics of the transmitted waves and in particular their acoustic intensity, and also different transducers as receivers, depending on the physical characteristics of the received waves.

The transducers 12 convert the echo waves into electrical signals. These electrical signals are processed to extract information about the biological tissue. This process creates an ultrasound image of the biological tissue from which these waves are reflected. The denser the organic tissue, the more the ultrasound waves will be reflected. Thus, the transducers will detect reflected sound when the ultrasound bounces off a tissue mass. Multiple transducers are used to image breast tissue.

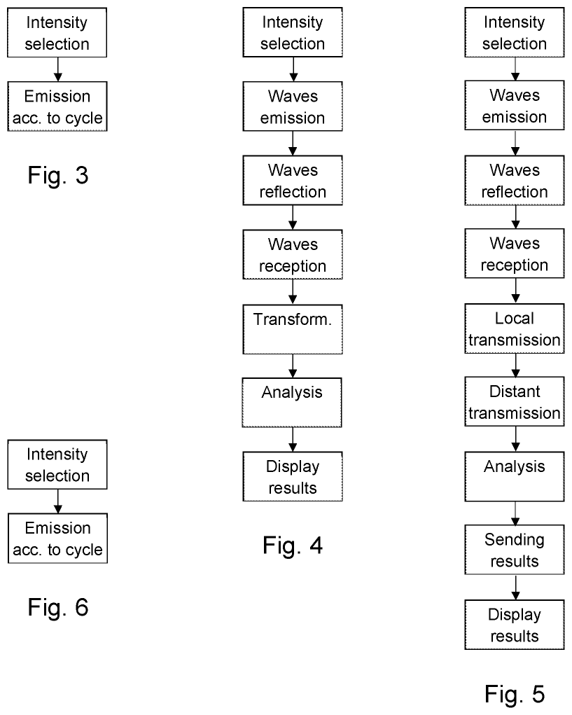

Three modes of use of the garment of the invention are described with reference to FIGS. 3 through 6. It should be noted that according to the present invention, the garment 10 allows for at least two of these three modes of use.

As is known, normal biological cells may undergo a process known as apoptosis. This is the process by which cells trigger their self-destruction in response to a signal. In the case of cancer cells, this process of apoptosis is not carried out and unhealthy cells, which would normally be eliminated by the body, are not destroyed. It has been shown that the application of ultrasound waves to biological tissue can have different effects, depending on the acoustic intensity of the ultrasound waves. In certain intensity ranges, ultrasound waves have the ability to regenerate the apoptosis process of cells.

The first described mode of use concerns the prevention and in particular the prevention of cancer of the breast or other part of the body on which the garment is placed.

In this mode of use illustrated by FIG. 3, the acoustic intensity of the ultrasound waves is first selected. This intensity is chosen between 0.05 and 2 W/cm². The frequency of the ultrasonic waves used can be chosen between approximately 600 kHz and 7 MHz. This frequency may be substantially the same in all modes of use or may depend on the mode of use, while preferably remaining in the range as defined above. The physical characteristics of the ultrasonic waves are chosen so as to be compatible with the function of regeneration of apoptosis.

Still with respect to prevention, the waves can be emitted according to a predefined cycle. A cycle that can be used in practice is, for example, sending ultrasound waves for a duration of 2 minutes, followed by a 15-minute break, the cycle being repeated, for example, 10 times during a day.

In principle, in the context of prevention, the transducers 12 of both cups 13 are used symmetrically and all transducers are used. Other embodiments are possible, such as using the transducers of only one cup at a time or using only a portion of the transducers.

A second embodiment illustrated in FIGS. 4 and 5 relates to the detection of abnormal cells and in particular cancerous cells. FIG. 4 illustrates a first embodiment of this mode of use. In this, the intensity of the ultrasonic waves is selected to be between 1 and 3 W/cm². The physical characteristics of the ultrasound waves are selected to be compatible with that encountered during an ultrasound examination.

Sound waves are emitted from the transducers 12, pass through the impedance matching element 20, and are transmitted to the user's biological tissues. Some of the emitted waves are reflected by these tissues, particularly by the interface between biological tissues having different densities.

The waves reflected by the user's cells are picked up by transducers 12 acting as receivers and are transformed into electrical signals by the transducers. These electrical signals are then transmitted to a processing unit 21 by the communication module 18 placed on the item of clothing.

According to a first embodiment, the processing unit 21 may also be placed directly on the item of clothing 10. However, this implies a suitable power supply and a size of the processing unit compatible with having it fixed on the item of clothing.

According to a second embodiment, the processing unit 21 is separate from the item of clothing. The processing unit 21 may be integrated with the external device 19, which can be a smartphone or a similar equipment comprising an application or a signal processing software and means for receiving the signals measured by the sensors of the item of clothing.

The processing unit 21 receives data from the external device 19, which has a receiver adapted to receive the signals transmitted by the communication module 18. These signals are then processed by the processing unit 21 in order to extract an information and/or an image.

When the data has been processed by the processing unit 21, a result can be made available to the user on the item of clothing by means of an audible alarm, a vibration or a light alarm indicating that the detector has not detected anything abnormal or indicating on the contrary that a doctor should be consulted.

The result can thus be made available on the external device 19, for example on a screen of the smartphone, as an image, text, sound or motion. It is also possible to display a result only if abnormal cells were detected during the processing of the measurements, and not to indicate anything if no abnormal cells were detected.

The information given to the user can therefore be the presence or absence of abnormal cells. This information can be delivered based on the comparison of the signals with information from a database stored for example in the processing unit 21. The image may be a three-dimensional image of the interior of the user's breasts, with, for example, clusters of abnormal cells being highlighted.

According to a third embodiment illustrated in FIG. 5, the steps of choosing the acoustic intensity, the emission of ultrasound waves by the transducers, the reflection of the ultrasound waves by the biological tissues and the reception of the waves by the receivers/transducers are the same as those described with reference to FIG. 4. The electrical signals from the receivers/transducers are also transmitted to an external device 19, which is preferably external to the garment 10.

In this embodiment, the external device 19 does not house a processing unit and does not necessarily have data processing means. On the other hand, this external device 19 has a remote communication channel with a remote processing center 22. In this case, the signals picked up by the transducers/detectors of the garment are transmitted locally to the external device 19. These signals are then transmitted remotely to the remote processing center 22 which takes over this processing.

As an example, the processing center 22 may form a 3D image of the cells and then compare this 3D image to a database of images corresponding to healthy and/or unhealthy cells. This comparison can be used to determine whether the cells of the person on which the measurements were made correspond to healthy or unhealthy cells. This comparison can typically be improved by using artificial intelligence and deep learning techniques.

It is also possible to store the images of each detection corresponding to a specific person and then compare the images with each other. In this way, it is possible to detect an evolution or a modification of the structure of the biological tissues for the person in question. If this evolution is abnormal or if the cells correspond to unhealthy cells, it is possible to warn the person to whom these images correspond and to recommend a medical visit.

It is also possible to collect data from a large number of people and associate them with the qualifier healthy or unhealthy, in order to form a knowledge base and gradually improve the reliability of image processing.

The abnormal cell detection phase can be done for example daily, weekly or at any chosen frequency. This phase can last from a few tens of seconds to a few minutes. A duration of two minutes is generally sufficient to perform a complete detection phase.

According to a third mode of use illustrated in FIG. 6, the garment is used for the treatment of a pathology such as cancer. In this mode of use, the intensity of the ultrasound waves is generally greater than 10 W/cm². The physical characteristics of the ultrasonic waves are chosen so as to allow the destruction of certain cells. The ultrasonic waves are not necessarily emitted by all the transducers, but can instead be emitted by targeted transducers, placed near areas where abnormal cells have been detected. These waves are emitted according to specific cycles that may depend on the location of the cells, their extension, a choice made by the doctor or other parameters. As an example, the treatment could include three 1-minute cycles, followed by 15-minute breaks, to be repeated five times during the day.

The item of clothing according to the invention allows at least two modes of use among prevention, detection and treatment. Preferably, the item of clothing allows for the detection mode of use and at least one from the prevention and treatment modes of use. The detection is initiated, for example, once a day, resulting in no indication if no abnormal cells have been detected and an alarm if abnormal cells have been detected. If no abnormal cells are detected, the prevention mode can be used, according to a predefined cycle.

If abnormal cells are detected, usually an alarm is triggered to indicate to the user that medical advice is recommended. In the event that treatment is required, the treatment mode can be activated on a predefined cycle. The detection mode makes it possible to monitor the evolution of the abnormal cells, also in case of treatment, and possibly to adapt the treatment accordingly. The adaptation of the treatment can concern the intensity of the waves used, the duration of a cycle, the number of cycles per time unit, the transducers activated, etc.

The mode of use to be implemented is chosen by selecting the acoustic intensity of the emitted ultrasonic waves. This intensity can be selected from at least two distinct intensity ranges, but preferably from three distinct intensity ranges. One range of intensities is intended to prevent the occurrence of abnormal biological cells such as cancer cells; one range is intended to detect abnormal biological cells; and one range is intended to eliminate such abnormal cells and treat the pathology.

As mentioned above, the intensity range for prevention is preferably between 0.05 and 0.2 W/cm². The intensity range for detection is preferably between 1 and 3 W/cm² and the intensity range for treatment is preferably greater than 10 W/cm².

If a sufficiently reliable knowledge base is built up, it is possible that the modes of use can be managed fully autonomously and automatically. This would mean that the detection phase is used to detect the presence or absence of abnormal cells, as explained above. If no abnormal cells are detected, the prevention phase is activated, also as described above. On the other hand, if abnormal cells are detected, the treatment phase is triggered autonomously, without necessarily seeking the intervention of medical personnel.

According to an advantageous implementation mode, the different modes of use are managed by means of an application running on an external device and/or by means of adapted software. In principle, different rights are assigned to different users. For example, the user is allowed to enter personal information and to log in securely to an account. However, in principle, the user is not allowed to change elements related to the various user cycles. They may be authorized to consult certain information.

The medical staff may be authorized to act on the parameters of the cycles governing the different modes of use. They may also be authorized to consult data such as stored images or information indicating the presence or absence of abnormal cells. The consultation or modification of parameters, values or other elements could be done either locally, for example by means of a wired connection or a short distance wireless connection such as Bluetooth or NFC, or on the contrary, remotely. This remote connection could be done through external devices such as smartphones or dedicated devices. It is clear that the security and confidentiality of the information passing through these devices must be taken into account.

The item of clothing 10 of the invention has been described in particular in the form of a bra. It could be made in many other forms, such as, for example, in any form of undergarment and in particular a blouse, a tank top, tights, briefs, etc. The item of clothing could also be made in the form of a visible garment or even possibly an overgarment such as a gaiter, pants, jacket, sleeve, etc. This item of clothing could be used as a preventive and/or curative measure against different forms of cancer such as breast cancer, testicular cancer, colon cancer, etc., without limitation to a particular type of cancer. The above-mentioned features in relation to an item of clothing in the form of a bra also apply in the case where the item of clothing is briefs or tights in particular.

The item of clothing may also be realized not as underwear, but as a visible garment. Such an embodiment could, for example, relate to a cap or hood, and be used in the prevention, detection and/or treatment of carcinomas.

The instruction receiver 15 also allows a person, who may be the user or a member of the nursing staff, to send instructions specific to the desired treatment.

The instruction receiver 15 may include a wireless receiving device or a connector such as a USB connector.

The measuring device 17 may be used to receive information regarding certain parameters of the user, such as for example a temperature of a certain area of the user's body. This can be done for example by means of a temperature sensor.

The result of these parameter measurements can be used, for example, by a member of the nursing staff to adapt, modify or validate treatment methods. These measurement results can also be taken into account automatically to adapt these treatment methods.

It is clear that this item of clothing could be combined with other conventional treatment mechanisms, especially in the field of cancer treatment.

1. An item of clothing intended to be worn by a user so as to cover a part of the body of this user, the item of clothing comprising:
a flexible clothing element covering said part of the body of the user;
an electrical signal generator; and
at least one transducer integral with the said flexible clothing element, said at least one transducer being arranged to convert said electrical signals from the electrical signal generator into ultrasounds and to transmit the ultrasounds generated by said transducer to said part of the user's body covered by said flexible clothing element;
wherein least one transducer is arranged to emit ultrasounds having at least two different acoustic intensities comprised in at least two distinct intensity ranges.

2. The item of clothing according to claim 1, wherein said item of clothing comprises at least two transducers of at least two different types, one of the types of transducers being arranged to emit ultrasounds having an intensity comprised in a first range of intensities and another type of transducers being arranged to emit ultrasounds having an intensity comprised in a second range of intensities distinct from said first range of intensities.

3. The item of clothing according to claim 1, wherein said at least one transducer is arranged to emit ultrasounds having three different intensities comprised in three distinct intensity ranges.

4. The item of clothing according to claim 1, wherein the ultrasounds of an intensity range have an acoustic intensity between 0.05 and 0.2 W/cm2.

5. The item of clothing according to claim 1, wherein the ultrasounds of an intensity range have an acoustic intensity between 1 and 3 W/cm2.

6. The item of clothing according to claim 1, wherein the ultrasounds of an intensity range have an acoustic intensity greater than 10 W/cm2.

7. The item of clothing according to claim 1, further comprising an impedance matching element disposed between said at least one transducer and said part of the user's body covered by said flexible clothing element.

8. The item of clothing according to claim 7, wherein the impedance matching element is a silicone element.

9. The item of clothing of claim 7, wherein the impedance matching element has an acoustic impedance between 1 and 20 MRayl.

10. The item of clothing according to claim 1, further comprising an instruction receiver arranged to receive signals for controlling the electrical signal generator.

11. The item of clothing according to claim 1, further comprising at least one receiver arranged to receive waves coming from said at least one transducer after reflection by said part of the user's body covered by said item of clothing.

12. The item of clothing according to claim 11, wherein said at least one receiver is formed by said at least one transducer.

13. The item of clothing according to claim 11, further comprising a communication module for transmitting data from said at least one receiver to an external device.

14. A method for monitoring biological cells of a user by means of an item of clothing intended to be worn by said user so as to cover a part of the body of this user, this item of clothing comprising a flexible clothing element covering said part of the body of the user, said item of clothing further comprising an electrical signal generator and at least one transducer integral with said flexible clothing element, said at least one transducer being arranged to convert said electrical signals from the electrical signal generator into ultrasounds and to transmit the ultrasounds generated by said transducer to said part of the user's body covered by said flexible clothing element, said method comprising:
selecting an acoustic intensity among at least two distinct acoustic intensity ranges;
supplying power to said at least one transducer so as to emit ultrasonic waves according to the selected acoustic intensity; and
emitting ultrasonic waves from said at least one transducer, said ultrasonic waves having an acoustic intensity within said selected intensity range.
